(19) United States
(12) Patent Application Publication
(10) Pub. No.: US 2006/0293698 A1
(43) Pub. Date: Dec. 28, 2006

(54) Retainer device for mitral valve leaflets

(75) Inventor: Nareak Douk, Lowell, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/169,989

(22) Filed: Jun. 28, 2005

(51) Int. Cl. A61B 17/128; A61F 2/24

(52) U.S. Cl. 606/142; 623/900

(57) ABSTRACT

A holding device for treating mitral valve regurgitation includes an anchoring portion and flexible arm portions. Upon deployment in the mitral valve, the arm portions assume a hook-shaped configuration and hold the leaflets in proximity to each other. One embodiment of the invention includes a method for preloading the holding device in a delivery catheter and deploying the device through the transverse axis of the mitral valve, allowing the arm portions of the holding device to grasp the mitral valve leaflets and hold them in proximity to each other, and thereby reduce both the cross sectional area of the mitral valve and regurgitation of blood through the valve.

### FIELD OF THE INVENTION

This invention relates generally to the treatment of mitral valve regurgitation and particularly to a method and device to reduce mitral valve regurgitation in a diseased heart.

### BACKGROUND

The heart is a four-chambered pump that moves blood efficiently through the vascular system. Blood enters the heart through the vena cava and flows into the right atrium. From the right atrium, blood flows through the tricuspid valve and into the right ventricle, which then contracts and forces blood through the pulmonic valve and into the lungs. Oxygenated blood returns from the lungs and enters the heart through the left atrium and passes through the mitral valve into the left ventricle. The left ventricle contracts and pumps blood through the aortic valve into the aorta and to the vascular system.

The mitral valve consists of two leaflets (anterior and posterior) attached to a fibrous ring or annulus. In a healthy heart, the mitral valve leaflets close during contraction of the left ventricle and prevent blood from flowing back into the left atrium. Due to various cardiac diseases, however, the mitral valve annulus may become distended causing the leaflets to remain partially open during ventricular contraction and thus allow regurgitation of blood into the left atrium. This results in reduced ejection volume from the left ventricle, causing the left ventricle to compensate with a larger stroke volume. However, the increased workload eventually results in dilation and hypertrophy of the left ventricle, further enlarging and distorting the shape of the mitral valve. If left untreated, the condition may result in cardiac insufficiency, ventricular failure, and ultimately death.

It is common medical practice to treat mitral valve regurgitation by either valve replacement or repair. Valve replacement involves an open-heart surgical procedure in which the patient's mitral valve is removed and replaced with an artificial valve. This is a complex, invasive surgical procedure with the potential for many complications and a long recovery period.

Mitral valve repair includes a variety of procedures to repair or reshape the leaflets to improve closure of the valve during ventricular contraction. If the mitral valve annulus has become distended, a frequent repair procedure involves implanting an annuloplasty ring on the mitral valve annulus. The annuloplasty ring generally has a smaller diameter than the annulus and, when sutured to the annulus, the annuloplasty ring draws the annulus into a smaller configuration, bringing the mitral valve leaflets closer together, and allowing improved closure during ventricular contraction.

Another approach to treating mitral valve regurgitation requires a flexible elongated device that is inserted into the coronary sinus and then undergoes a change that causes it to assume a reduced radius of curvature and, as a result, causes the radius of curvature of the coronary sinus and the circumference of the mitral annulus to be reduced. A similar elongated device may be inserted into the coronary sinus and anchored at each end. The length of the elongated device is adjusted to reduce the curvature of the coronary sinus and thereby change the configuration of the mitral annulus. Due to the nature of the anchors, this device may cause significant damage to the coronary sinus and surrounding cardiac tissue. Also, leaving a device in the coronary sinus may result in formation and breaking off of thrombus that may pass through the right atrium, the right ventricle, and ultimately to the lungs causing a pulmonary embolism.

Another approach to reducing mitral valve regurgitation requires suturing or clipping the mitral valve leaflets together. Sometimes called a “bow-tie” repair, this method may be performed using minimally invasive surgical techniques, and comprises grasping or otherwise stabilizing the leaflets, piercing each leaflet with either suture material or a metallic clip, and fastening the center of the leaflets together. Some devices do not pierce the leaflets, but grasp and pinch them tightly together. Blood flows through the two side openings thus formed, and regurgitation is reduced. However, all of these devices cause damage to the leaflets and, potentially, permanent scaring and deformity of the leaflets.

It would be desirable, therefore to provide a method and device for reducing mitral valve regurgitation that would be easily delivered using minimally invasive surgical techniques, and further, would overcome the limitations and disadvantages inherent in the devices and methods described above, especially the need to pierce the mitral valve leaflets and the potential for damage to the leaflets and other cardiac tissues.

### BRIEF SUMMARY OF THE INVENTION

One aspect of the invention provides a system for treating mitral valve regurgitation comprising a delivery catheter and a holding device. The holding device comprises two arm portions and a central anchoring portion. The holding device may be positioned in a catheter delivery configuration and inserted into the delivery catheter. When deployed from the delivery catheter, the holding device assumes a deployment configuration in which the arm portions are hook-shaped. The hook-shaped arms contact the leaflets of the mitral valve and hold the leaflets in proximity to each other and thereby reduce regurgitation of blood through the mitral valve.

Another aspect of the invention provides a holding device for treating mitral valve regurgitation comprising at least two flexible arm portions and a central anchoring portion. The holding device can be positioned in a catheter delivery configuration and a deployment configuration. When the device is deployed from the delivery catheter, the arm portions assume a hook-shaped configuration and contact the anterior and posterior leaflets of the mitral valve. The arm portions hold the leaflets in proximity to each other and thereby reduce regurgitation of blood through the mitral valve.

Another aspect of the invention provides a method for treating mitral valve regurgitation and includes providing a holding device comprising at least two flexible arm portions extending from a central anchoring portion, positioning the holding device in a catheter delivery configuration, and preloading the holding device into the internal lumen of a delivery catheter. The distal portion of the delivery catheter is advanced through the vascular system and into the left atrium. The distal tip of the catheter is positioned in the transverse axis of the mitral valve leaflets and the holding device is deployed from the catheter. Once deployed, the arm portions of the holding device assume a hook-shaped configuration and contact the mitral valve leaflets, drawing and holding the leaflets into proximity to each other.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The drawings are not to scale. The foregoing aspects and other attendant advantages of the present invention will become more readily appreciated by the detailed description taken in conjunction with the accompanying drawings.

### BRIEF DESCRIPTION OF THE FIGURES

FIG. 1 is a cross sectional schematic view of a heart showing the location of the mitral valve;

FIG. 2 shows one embodiment of a holding device in a deployment configuration, in accordance with one aspect of the invention;

FIG. 3 shows another embodiment of a holding device in a deployment configuration in accordance with one aspect of the invention;

FIG. 4 shows the holding device of FIG. 2 in a delivery configuration, in accordance with one aspect of the invention;

FIG. 5 shows the holding device of FIG. 3 in a delivery configuration, in accordance with one aspect of the invention;

FIG. 6 is a cross sectional side view of one embodiment of a delivery system for delivering a holding device in accordance with another aspect of the invention;

FIG. 7 is a schematic view illustrating the placement of the holding device in the transverse axis of the mitral valve, in accordance with another aspect of the invention;

FIG. 8 is a cross sectional longitudinal schematic view of the human heart with the holding device positioned in the transverse axis of the mitral valve, in accordance with another aspect of the invention; and

FIG. 9 is a flow diagram of a method of treating mitral valve regurgitation, in accordance with one aspect of the invention.

### DETAILED DESCRIPTION

Throughout this specification, like numbers refer to like structures.

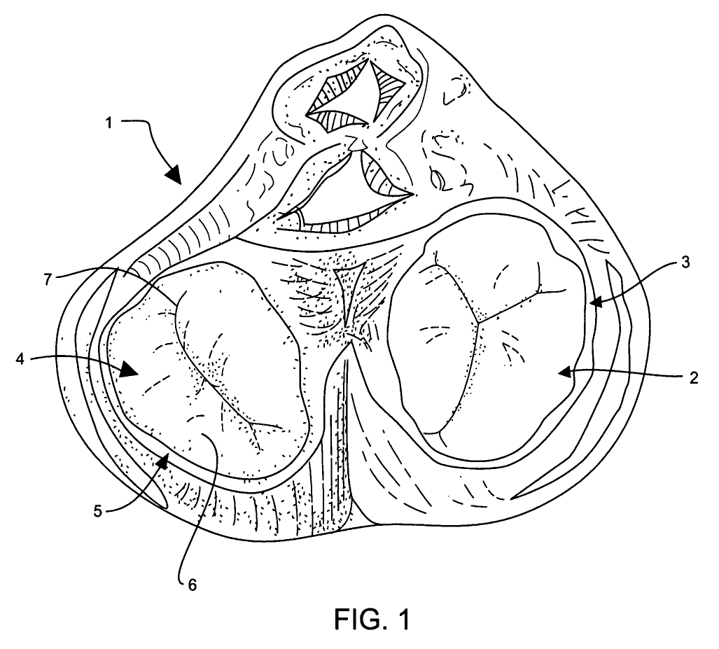

Referring to the drawings, FIG. 1 shows a cross-sectional view of heart 1 having tricuspid valve 2 and tricuspid valve annulus 3. Mitral valve 4 is adjacent mitral valve annulus 5. Mitral valve 4 is a bicuspid valve having anterior cusp 7 and posterior cusp 6. Anterior cusp 7 and posterior cusp 6 are often referred to, respectively, as the anterior and posterior leaflets.

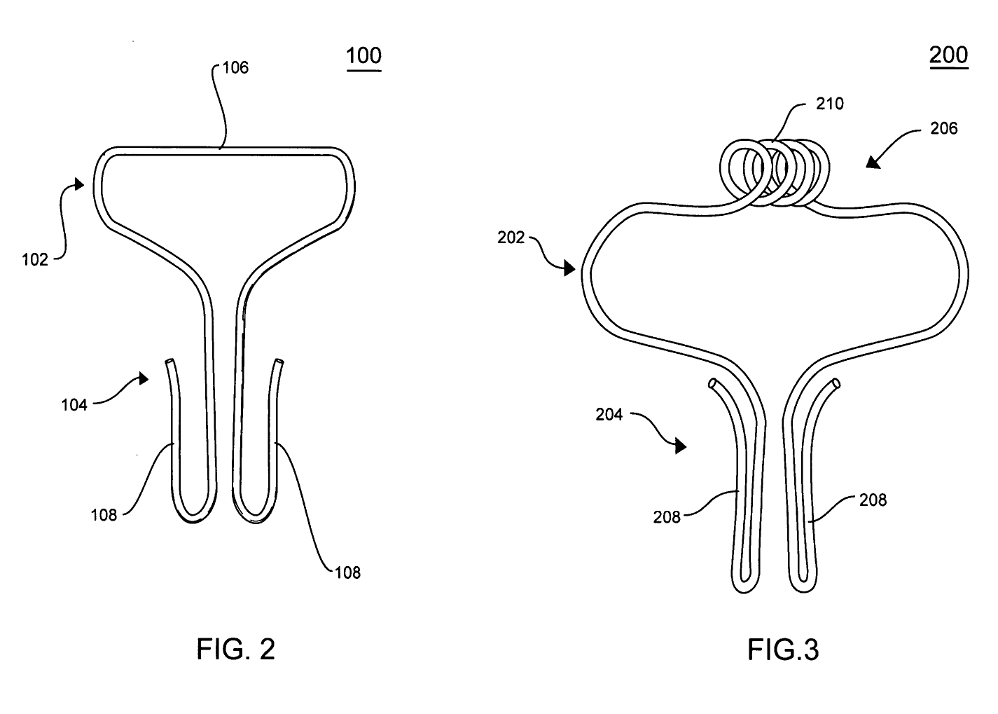

FIG. 2 portrays a holding device 100 for treating mitral valve regurgitation. Holding device 100 includes central anchoring portion 102, and at least two arm portions 104 extending from anchoring portion 102. Central anchoring portion 102 is circular or elliptical in shape, and has top portion 106 that is of a size so that when device 100 is placed adjacent to mitral valve 4, anchoring portion 102 will lodge against mitral valve annulus 5, and hold device 100 in place. Holding device 100 may be positioned so that when anchoring portion 102 is seated firmly against mitral valve annulus 5, arm portions 104 extend through the transverse axis of mitral valve 4. Distal segments 108 of arm portions 104 are hook-shaped and sized to grasp mitral valve leaflets 6 and 7 and hold leaflets 6 and 7 in proximity to each other when device 100 is deployed through the transverse axis of mitral valve 4.

FIG. 3 portrays holding device 200 having an alternative configuration, in accordance with one aspect of the invention. Holding device 200 has central anchoring portion 202 and arm portions 204, similar to holding device 100. Additionally, device 200 has coil portion 210 attached to top portion 206. In one embodiment of the invention, coil portion 210 gives holding device 200 added flexibility in order to facilitate preloading device 200 into the lumen of a catheter and delivering the device adjacent to mitral valve annulus 5. As described above, distal segments 208 of arm portions 204 are in a hook-shaped configuration when deployed adjacent to mitral valve annulus 5.

Holding devices 100 and 200 are made of a flexible, biocompatible material that has “shape memory” so that either holding device 100 or 200 can be extended into an elongated configuration and inserted into a delivery catheter, but will re-assume its original shape and dimensions when deployed adjacent to the mitral valve annulus. In one embodiment of the invention, holding devices 100 and 200 comprise nitinol, a biocompatible material that gives the devices the needed flexibility and shape memory. Fabrication of either holding device 100 or 200 includes forming the device from nitinol wire or thick-walled nitinol tubing, and using heat to set the nitinol in the configuration shown in FIG. 2 or FIG. 3. It is desirable that the holding device be radiopaque to facilitate placement of the device. In one embodiment of the invention, the holding device comprises thick-walled nitinol tubing filled with a radiopaque material such as gold, tungsten, silver, iridium, or platinum. In addition, the surface of holding device 100 or 200 is preferably hemocompatible, and causes minimal blood clotting or hemolysis when exposed to flowing blood. Dacron® polyester fiber (E.I. Du Pont De Nemours & CO., Inc) is a material known in the art to have the necessary hemocompatible properties, and may be used in the cardiovascular system. In one embodiment of the invention, holding device 100 or 200 is covered with a Dacrone® polyester fiber cover.

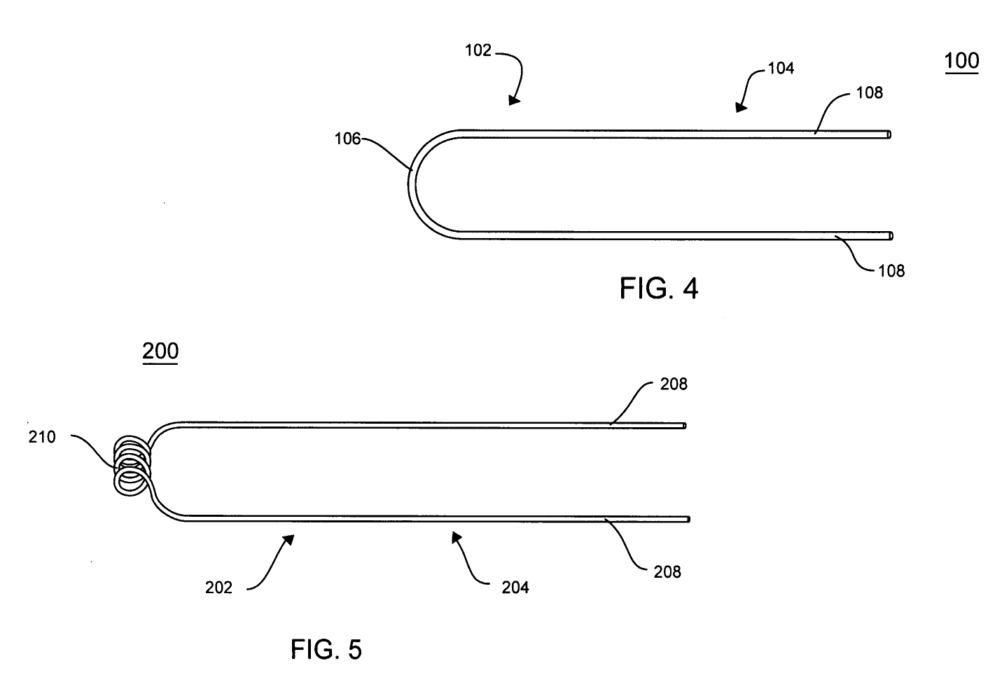

FIG. 4 and FIG. 5 portray holding devices 100 and 200, respectively, in an elongated, catheter delivery configuration. The top portion 106 of device 100 has been drawn away from anchoring portion 102 of the device until portion 102 is in an elongated, essentially linear configuration. In addition, distal portions 108 of arms 104 have been extended to the linear delivery configuration. Similarly, in the delivery configuration, portions 202 and 206 of holding device 200 (FIG. 5) have been drawn into an essentially linear configuration and distal arm portions 208 have been extended. Coiled portion 210 gives added flexibility to anchoring portion 202 to facilitate positioning the device in the catheter delivery configuration and inserting the device into a delivery catheter lumen. Because holding devices 100 and 200 comprise shape memory material such as nitinol, holding devices 100 and 200 will spontaneously revert to their unconstrained, deployed configurations shown in FIG. 2 and FIG. 3, respectively, when free to do so.

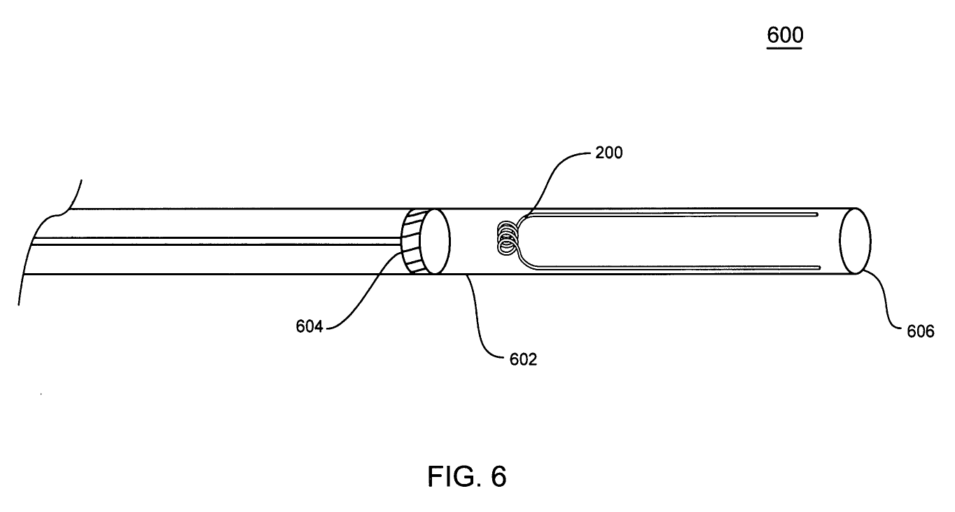

FIG. 6 is a cross sectional side view of the distal portion of system 600 for treating mitral valve regurgitation using minimally invasive surgical techniques. In one embodiment of the invention, flexible holding device 200 is placed in its catheter delivery configuration (FIG. 5) and inserted into the distal portion of the interior lumen of the delivery catheter 602. In one embodiment of the invention, delivery catheter 602 is flexible and configured so that it can be inserted into the cardiovascular system of a patient. Such catheters are well known in the art and are, for example, between 8 and 24 French in diameter, and include an exterior wall or sheath made of flexible, biocompatible polymeric materials such as polyurethane, polyethylene, nylon and polytetrafluoroethylene (PTFE). Within the interior lumen formed by the sheath of catheter 602, and proximal to holding device 200 is a deployment device, such as a flexible rod 604 that is used to deploy holding device 200 by pushing it from distal tip 606 of catheter 602. After holding device 200 is deployed, flexible rod 604 may be withdrawn from catheter 602. In one embodiment of the invention, the interior surface of catheter 602 is coated with a lubricious material such as silicone, polytetrafluoroethylene (PTFE), or a hydrophilic coating. The lubricious interior surface of catheter 602 facilitates the longitudinal movement of flexible rod 604 and deployment of holding device 200.

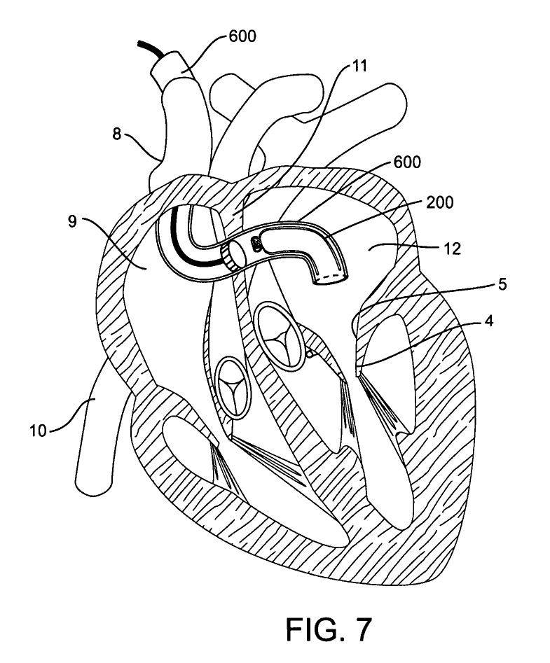

To deliver holding device 100 or 200 adjacent to mitral valve 4 (FIG. 7), delivery catheter 600 containing either holding device 100 or 200 may be inserted into either the jugular vein, or the subclavian vein, through superior vena cava 8, and into right atrium 9. Alternatively, catheter 600 may be inserted through inferior vena cava 10 and into right atrium 9. Transeptal wall 11 between right atrium 9 and left atrium 12 is then punctured with a guide wire or a guide catheter with a sharp tip and the distal end of delivery catheter 600 is advanced into left atrium 12 and placed in proximity to annulus 5 of mitral valve 4. In one embodiment of the invention, the placement procedure is performed using fluoroscopy, echocardiography, intravascular ultrasound, angiography, or other means of visualization.

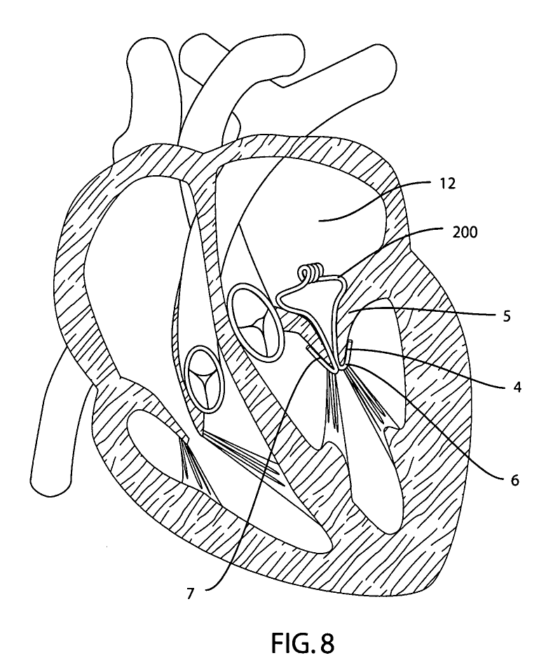

FIG. 8 is a schematic longitudinal sectional view of the heart showing holding device 200 deployed in the transverse axis of mitral valve 4, in accordance with one embodiment of the invention. Anchoring portion 202 of device 200 is in left atrium 12 of the heart, seated firmly against mitral valve annulus 5. Flexible arm portions 204 extend through the transverse plane of mitral valve 4, and hook shaped distal portions 208 grasp leaflets 6 and 7, and hold them in proximity to each other.

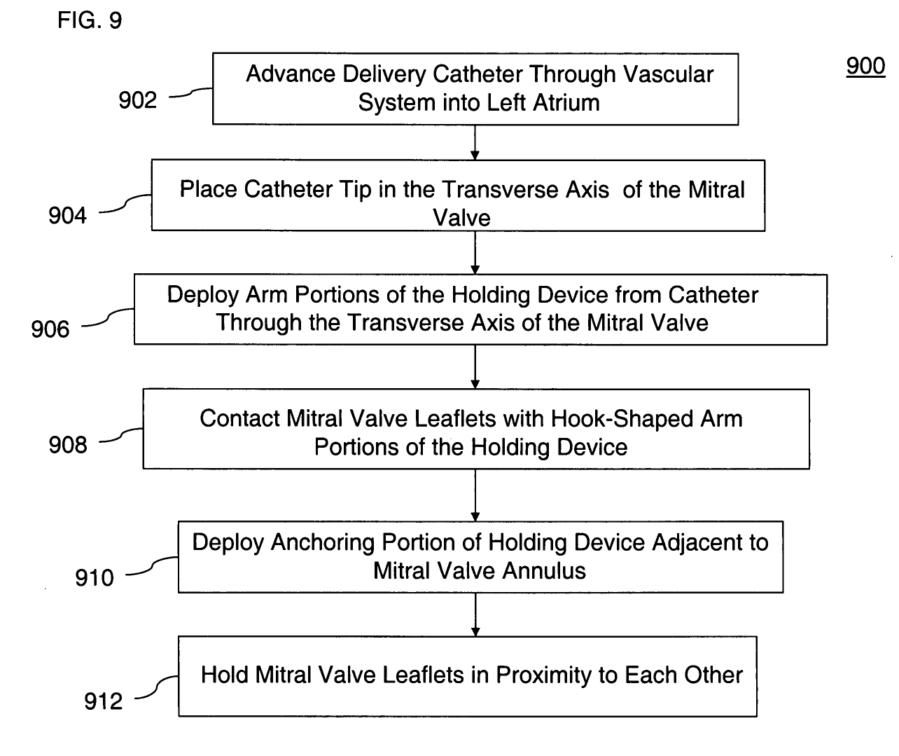

FIG. 9 is a flowchart illustrating a method 900 for treating mitral valve regurgitation, in accordance with one embodiment of the invention. As described in FIG. 7, the distal tip of delivery catheter 602 containing holding device 200 is advanced through the vascular system of the patient, passed through right atrium 9, and into left atrium 12, adjacent to mitral valve annulus 5 (Block 902). Next, distal tip 606 of catheter 602 is carefully placed in the transverse axis of mitral valve 4 (Block 904). Arm portions 204 of holding device 200 are then deployed from the interior lumen of catheter 602 through the transverse axis of mitral valve 4 (Block 906). A deployment device, such as flexible rod 604 within catheter 602 is used to deploy holding device 200 by pushing it through distal tip 606 of catheter 602 and into the transverse axis of mitral valve 4. When extruded from catheter 602, distal arm portions 208 of holding device 200 will automatically assume a hook shaped configuration. The hooked portions of arms 208 will grasp leaflets 6 and 7 of mitral valve 4 (Block 908) and draw leaflets 6 and 7 toward the center of the transverse axis of mitral valve 4. Anchoring portion 202 of holding device 200 may then be deployed from distal tip 606 of delivery catheter 602 adjacent to mitral valve annulus 5, and allowed to assume an elliptical configuration, causing anchoring portion 202 of device 200 to seat firmly against annulus 5. Arm portions 208 of holding device 200 will hold mitral valve leaflets 6 and 7 in proximity to each other (Block 910). Blood will flow through openings on either side of the clipped portion of leaflets 6 and 7, but, because the cross sectional area of mitral valve 4 will be reduced, less regurgitation of blood back into left atrium 12 will occur. Improved mitral valve function can be evaluated from decreases in pressure in left atrium 12. Finally, delivery catheter 602 may be withdrawn from the body of the patient.

While the invention has been described with reference to particular embodiments, it will be understood by one skilled in the art that variations and modifications may be made in form and detail without departing from the spirit and scope of the invention.

What is claimed is:

1. A system for treating mitral valve regurgitation in a heart comprising:
a delivery catheter;
a holding device insertable within the catheter, the holding device comprising first and second arm portions and a central anchoring portion, wherein the holding device is positionable in a catheter delivery configuration, and in a deployment configuration, and when the device is deployed from the delivery catheter the arm portions assume a hook-shaped configuration, contact an anterior and a posterior leaflet of the mitral valve, and hold the leaflets in proximity to each other to reduce regurgitation of blood through the mitral valve.

2. The system of claim 1 wherein each arm portion of the holding device is substantially linear in the catheter delivery configuration and hook-shaped in the deployment configuration.

3. The system of claim 1 wherein the holding device is comprised of a shape-memory material and the arm portions assume the hook-shaped configuration when deployed from the catheter.

4. The system of claim 3 wherein the shape-memory material is nitinol.

5. The system of claim 1 wherein the holding device comprises a continuous strand of wire.

6. The system of claim 5 wherein the continuous strand of wire is hollow and forms an interior lumen.

7. The system of claim 6 wherein the interior lumen is filled with a radiopaque material selected from the group consisting of gold, tungsten, silver, iridium, and platinum.

8. The system of claim 1 wherein the anchoring means comprises a bent portion of the holding device, the bent portion forming a circular or elliptical configuration sized to contact the mitral valve annulus and anchor the device in the transverse axis of the mitral valve leaflets.

9. The system of claim 1 wherein the delivery catheter comprises:
an outer sheath;
a delivery lumen within the sheath at a distal end of the catheter; and
a deployment device positioned within the lumen, wherein when the system is delivered through the vascular system and adjacent to a mitral valve, the holding device is inserted through the transverse axis of the mitral valve.

10. A holding device for treating mitral valve regurgitation in a heart comprising:
at least first and second arm portions; and
a central anchoring portion, wherein the holding device is positionable in a catheter delivery configuration and a deployment configuration and when the device is deployed from the delivery catheter the arm portions assume a hook-shaped configuration, contact an anterior and a posterior leaflet of the mitral valve, and hold the leaflets in proximity to each other to reduce regurgitation of blood through the mitral valve.

11. The device of claim 10 wherein each arm portion of the holding device is substantially linear in the catheter delivery configuration and hook-shaped in the deployment configuration.

12. The device of claim 10 wherein the holding device is comprised of a shape-memory material and the arm portions assume a hook-shaped configuration when the holding device is deployed from the catheter.

13. The device of claim 12 wherein the shape-memory material is nitinol.

14. The device of claim 10 wherein the holding device comprises a continuous strand of wire.

15. The device of claim 14 wherein the continuous strand of wire is hollow and forms an interior lumen.

16. The device of claim 15 wherein the interior lumen is filled with a radiopaque substance selected from the group consisting of gold, tungsten, silver, iridium, and platinum.

17. The device of claim 10 wherein the anchoring means comprises a bent portion of the holding device, the bent portion forming a circular or elliptical configuration sized to contact the mitral valve annulus and anchor the clip in the transverse axis of the mitral valve leaflets.

18. A method of treating mitral valve regurgitation, the method comprising:
providing a holding device including at least two arm portions flexibly extending from a central anchoring portion;
preloading the holding device in a catheter delivery configuration into a lumen of a delivery catheter;
advancing the delivery catheter through the vascular system and into the left atrium;
positioning the distal tip of the catheter in a transverse axis of the mitral valve leaflets;
deploying the holding device from the catheter and allowing the arm portions to assume a hooked-shape;
contacting the leaflets with the hooked shaped arm portions; and drawing the leaflets in proximity to each other.

19. The method of claim 18 further comprising:
positioning the anchoring portion against the mitral valve annulus.

20. The method of claim 18 wherein drawing the mitral valve leaflets into proximity to each other reduces mitral valve regurgitation.
